(19) United States
(12) Patent Application Publication
(10) Pub. No.: US 2013/0096610 A1
(43) Pub. Date: Apr. 18, 2013

(54) BIOPOLYMER MICROTHREADS WITH MICROSCALE SURFACE TOPOGRAPHIES

(71) Applicants: George Pins, Holden, MA (US); Shawn Carey, Ithaca, NY (US)

(72) Inventors: George Pins, Holden, MA (US); Shawn Carey, Ithaca, NY (US)

(21) Appl. No.: 13/635,500

(22) PCT Filed: Mar. 17, 2011
(86) PCT No.: PCT/US2011/028909

§ 371 Date: Jan. 2, 2013

Related U.S. Application Data
(60) Provisional application No. 61/314,829, filed on Mar. 17, 2010.

(51) Int. Cl. D06M 10/00; A61L 17/00

(52) U.S. Cl. CPC D06M 10/00; A61L 17/00
USPC 606/228; 424/400; 424/93.1; 34/284; 206/570; 206/572; 428/400

(57) ABSTRACT

In one aspect, the invention features a textured microthread that includes or is fashioned from a naturally occurring polymer and that has a surface comprising micron-scale ridges with intervening grooves.

### RELATED APPLICATIONS

This application claims the benefit of the filing date of U.S. Provisional Application No. 61/314,829, which was filed on Mar. 17, 2010. For the purpose of any U.S. application or patent that claims the benefit of U.S. Provisional Application No. 61/314,829, the content of that earlier filed application is hereby incorporated by reference in its entirety.

### FIELD OF THE INVENTION

This invention relates to tissue engineering and more particularly to textured microthreads that can be used to study phenomena such as cell migration in vitro or to augment or repair damaged or defective tissue in vivo.

### BACKGROUND

Tissue engineering is a field that seeks to mimic the natural biological processes of tissue development and regeneration to produce functional biological constructs that restore, maintain or improve native tissue (Langer and Vacant, Science 260:920-926 (1993)). While the goal of tissue engineering has remained largely unchanged since its inception, consistent developments have advanced the field from basic cultures of cells to the fabrication of complex, precisely engineered tissue constructs that are structurally and functionally analogous to native tissue. This shift can be partially attributed to an increased understanding of the importance of cell-matrix interactions. Throughout the body, the biochemical and mechanical interplay between cells and their underlying matrix are of paramount importance to the development, functioning, and repair of tissue. Therefore, in building engineered tissue constructs, which are fabricated by coupling isolated tissue-specific cells with biomaterial scaffolds, we seek to recreate the natural conditions of the cellular microenvironment as closely as possible. To achieve this, the biochemistry and surface topography of the biomaterial scaffold have been precisely controlled, which influences cellular attachment, proliferation, and ordered development (Linnes of al., Biomaterials. 28:5298-5306 (2007)).

### SUMMARY

The present invention is based, in part, on our development of textured microthreads. As the name implies, the overall appearance of these compositions is thread-like; they have an elongated structure (i.e., one that is longer than it is wide), which may be essentially circular in a cross-section taken perpendicular to the long axis. The microthreads are textured in that their outer surfaces are contoured. Further, the textured nature of the microthreads is such that one would most naturally measure and/or express the dimensions of the surface contours in microns. The textured surface can be formed from a series or plurality of micron-scale ridges with intervening grooves. A majority of the ridges (e.g., the ridges covering more than half and up to essentially all of the surface of the microthread) can be aligned with respect to one another, and the aligned ridges can be oriented with the long axis of the microthread (i.e., the ridges can run parallel to the long axis of the microthread). As will be appreciated, while some of the ridges may be perfectly aligned with respect to one another and while some of the aligned ridges may be perfectly oriented with the long axis of the microthread, the present compositions are not so limited. The topography may resemble the gills of a mushroom and, when viewed with the aid of an electron microscope, it may be seen that only a certain percentage of the ridges (e.g., about 40-80% of the ridges) run substantially parallel to one another. A representative topography is shown in FIG. 2.

Accordingly, in one aspect, the invention features a textured microthread that includes or is fashioned from a naturally occurring polymer and that has a surface comprising micron-scale ridges with intervening grooves. The microthread can have a diameter of about 20 microns to about 500 microns (e.g., about 20-100 μ; about 100-200 μ; about 200-250 μ; or about 250-500 μ, and the ridges can extend from the surface of the microfiber to a height of about 0.5 microns to about 20 microns (e.g., about 0.5-2.0 μ; about 0.5-5.0 μ; about 1.0-10.0 μ; about 10-20 μ; or about 2.0, 3.0, 5.0, 10, 12, 15, or 18 μ). As noted, there may be some variability in the height of the individual ridges, and the height can therefore be expressed as the average height of the ridges or the height of a certain substantial percentage of the ridges. For example, where the surface topography includes ridges of about 10 μ in height, at least or about 50%, 60%, 75%, 80%, 85%, 90%, 95%, or 99% of the ridges can be at least or about 10 μ in height. With respect to width, the ridges can be about 0.5 microns to about 20 microns in width (e.g., about 0.5-2.0 μ; about 0.5-5.0 μ; about 1.0-10.0 μ; about 10-20 μ; or about 2.0, 3.0, 5.0, 10, 12, 15, or 18 μ). As with height, the width may be the average width or a width attained by a substantial percentage of the ridges. The width can be measured at the base of the ridge and/or at its widest point.

The tensile strength can be increased relative to a comparable microthread that has not been texturized. For example, the tensile strength of a textured microthread can be at least or about 20-500% greater than that of a comparable microthread that has not been texturized (e.g., by the methods described herein). In one embodiment, the textured microthread can have a tensile strength of about 1.0 to about 10.0 MPa (e.g., about 2.0 MPa to about 4.0 MPa; e.g., about 1.0, 2.0, 3.0, 4.0, 5.0, 6.0, 7.0, 8.0, 9.0, or 10,0 MPa). Our experiments to date indicate that freezing the microthreads at −80° C. approximately doubles their ultimate tensile strength. We have consistently observed this increase in mechanical strength.

A variety of polymeric materials, used singly or in combinations thereof, can be used to fashion the microthread. The polymer can be a naturally occurring polymer, a synthetic polymer, or a combination thereof. Where the textured microthread includes a naturally occurring polymer, the polymer can be a proteoglycan (e.g., heparin sulfate, chondroitin sulfate, or keratin sulfate), a protein, polypeptide, or a glycoprotein (e.g., collagen, silk, fibrinogen, elastin, tropoelastin, fibrin, fibronectin, or gelatin) or a carbohydrate or a polysaccharide (e.g., hyaluronan, a starch, alginate, pectin, cellulose, chitin, or chitosan). The protein can be a full-length, naturally occurring protein and the polypeptide can be a fragment thereof or a short, naturally occurring or synthetic polymer of amino acid residues (e.g., a polymer of about 10-50 amino acid residues). In combination, for example, the microthread can include collagen and a proteoglycan and/or a glycoprotein (e.g., collagen and chondroitin sulfate proteoglycan).

Further, any of the textured microthreads described herein can be associated with a therapeutic agent. When associated, the microthread carries the therapeutic agent, whether the force is by an electrostatic interaction, adsorption, or a molecular bond (e.g., a covalent bond). There are a number of techniques that can be used to embellish the microthreads. We can, for example, crosslink the microthreads with agents such as glutaraldehyde, carbodiimide, and transglutaminase (factor XIII). The microtheads can also undergo dehydrothermal (DHT) treatment in which heat and a vacuum are applied together. These treatments can be used alone or in combination. As noted, therapeutic agents can be passively or strategically conjugated to the surfaces of the microthreads. For example, we have passively adsorbed fibronectin to microthreads, mixed FGF-2 into the threads during processing, and a host of molecules can be tethered to the surface.

The therapeutic agent can be a biological cell (e.g., a stem cell, whether derived from an embryonic or adult organism), a small organic compound, or a biological agent. In addition to stem cells and other pluripotent or progenitor cells, the associated biological cell can be a fibroblast, chondrocyte, osteocyte, myocyte, neuron, glial cell, hepatocyte, epithelial cell, dermal cell, renal cell, or adipocyte. Essentially any cell can be used, and the cell(s) selected may be of the same type as the cell(s) present in the tissue to be treated. Suitable small organic compounds include vitamins, antioxidants, anti-viral agents, anti-fungal agents, antibiotics, anti-inflammatory agents, and chemotherapeutic agents.

The biological agent can be a nucleic acid or protein (e.g., a protein classified as a cytokine, growth factor, cellular ligand, cellular receptor, enzyme, or an antibody). In addition to conventional tetrameric antibodies, the textured microthreads can be associated with an antibody fragment, such as an Fab, F(ab′), or F(ab′)₂ fragment, or with a single chain antibody (scFv). The biological agent can also be an extracellular matrix component.

As noted, the textured microthreads can be associated with a protein that serves as a therapeutic agent. A first type of protein can also be adsorbed to an outer surface of the microthread and, if desired, a second type of protein can then be adsorbed, conjugated, or fused to the first protein; the first protein acting as a coating or linker between the microthread and the second (e.g., therapeutic) protein. The protein can be fibronectin or one or more fragments of fibronectin.

The microthreads described herein can be braided and, whether braided or not can further include a cladding material, glue (e.g., fibrin glue), binder (e.g., collagen gel), suture, or sleeve, any of which may bundle the microthread together with other microthreads. Thus, the invention encompass bundles of textured microthreads (having, for example, about 3-30 microthreads). Thus, the microthreads can be braided together, bunded or otherwise bound together for delivery (e.g., to a tissue culture environment or a patient's tissue). For example, the threads can be gathered and grouped together with a suture (e.g., a silk suture) or other cladding material. It has been shown that bundled structures of silk fiber scaffolds increase surface area for cell attachment and ECM deposition while minimizing mass transfer limitations (Altman et al., Biomaterials. 23:4131-4141 (2002)), and we expect the bundled microthreads of the present invention to have an increased surface area and facilitate cell attachment and/or ECM deposition. The microthreads, alone or when bundled, can also be fashioned into a mesh, lattice, or other scaffold.

In another aspect, the present invention features methods of making a textured microthread. The methods can include the steps: (a) providing a microthread; (b) drying and subsequently rehydrating the microthread, thereby generating a rehydrated microthread; and (c) freeze-drying the rehydrated microthread, thereby forming a textured microthread. Further, the method can include step (d): lyophilizing the frozen microthread, thereby exposing the surface of the textured microthread. For example, collagen and/or fibrin threads can be dried using processes known in the art, and the dried threads can be mounted under tension on a frame (such as a PDMS frame). The mounted threads can then be hydrated for about one hour in a container (e.g., an aluminum container) containing distilled water or any of a number of other aqueous solutions (e.g., PBS, a cell culture medium, or an aqueous medium containing a surfactant such as Tween-20 or DMSO). The hydrated threads and hydrating solution along with the containers are transferred to a controlled temperature freezer (e.g., −20° C., −80° C.) for at least or about two hours. The frozen systems (including the microthreads, the solution, and the container) are then transferred to a shelf in a lyophilizer (e.g., at −45° C.). After about two hours, the vacuum in the lyophilizer is lowered to 50 mtorr, and the shelf temperature is ramped to 0° C. The threads are lyophilized under these conditions for about 12-24 hours. After lyophilization, the dried grooved threads are removed from the frames and stored in a desiccator until use.

In the context of the production methods, the microthreads can be fashioned from the same materials as described above or elsewhere herein (e.g., a naturally occurring polymer such as collagen).

The rehydrating step can be carried out by exposing a dried microthread to a buffered solution, and the freeze-drying step can be carried out at a temperature between about −20° C. and −200° C.

The methods as described herein can further include step (e): associating a therapeutic agent with the surface of the textured microthread, and the therapeutic agent can be of the type described above or further below (e.g., a biological cell).

The invention includes a textured microthread made by a method as described herein as well as tissue engineering constructs and physiologically acceptable compositions including the textured microthreads as described herein. The physiologically acceptable compositions can include, in addition to the textured microthreads, a physiologically acceptable carrier (e.g., a solution such as a buffered solution (e.g., PBS) or gel (e.g., a hydrogel)). The solution can also be a tissue culture medium, as the textured microthreads are useful in assaying cell migration (e.g., in the context of potential inhibitors of metastases).

Also within the scope of the present invention are kits comprising the textured microthreads described herein and/or tissue engineering constructs including them. For example, the kit can include a textured microthread or a tissue engineering construct, optionally packaged within a sterile container, and one or more of: a package insert comprising instructions for use, an antiseptic agent, a buffered solution, a saline solution, a gel, an ointment, a cream, scissors, a scalpel, a clamp, a needle, a spatula, sutures, gauze, surgical gloves, and a tissue culture vessel.

Also featured are uses; use of the textured microthread in the preparation of a medicament and use of the textured microthread in the preparation of a medicament for tissue repair. For example, the use can feature tissue repair comprising tissue augmentation or tissue replacement (e.g., of the skin, muscle, bone, or a connective tissue). The tissue repair may be necessitated by a traumatic injury, a surgical procedure, a congenital malformation, or a disease, disorder, or condition that results in tissue loss, malfunction, or malformation.

Also featured are methods of treatment: a method of treating a patient in need of tissue augmentation or repair. The methods include administering to the patient a textured microthread as described herein or a tissue engineering construct containing such microthreads. For example, the microthread can be placed in contact with the tissue in need of augmentation or repair. In any instance, the patient can be a human patient, but the invention is not so limited. Veterinary uses and methods are also within the scope of the present invention.

The textured microfibers of the present invention may have one or more of the following characteristics. They may be biodegradable, and their rate of degradation may match that of new tissue deposition. They may be biocompatible through all stages of degradation. Their mechanical properties may be analogous to those of native tissue throughout the regeneration process, and they may be biofunctional (e.g., having the ability to support the proliferation and differentiation of both implanted and native cells, capable of ECM secretion, and capable of forming functional tissue). Further, the textured microthreads may have the ability to enhance any of the above attributes (through, for example, composite structures, crosslinking, surface chemistry modifications, association with biologically active molecules and cells on their surfaces, and the like).

We expect the present compositions to have uses and advantages over prior compositions generated by freeze-drying. For example, in hydrogels, phase separation via freeze-drying (lyophilization) takes advantage of the biphasic solid-liquid system: a polymer lattice is hydrated with a solvent, such as water, and the hydrated network is subsequently solidified (frozen). The polymer is localized between growing ice crystals, forming a continuous interpenetrating network of polymer and ice crystals. The structure is then sublimated, leaving behind a pore structure that mirrors the ice crystals (O'Brien et al., Biomaterials. 25:1077-1086 (2004)). Controlling the freezing conditions (including the solvent and freezing rate) allows for precise control over final pore structure (Schoof et al., J Biomed. Mater. Res. 58:352-357 (2001). While sponges created from natural biological polymers using this technique provide essential three-dimensional support and signaling templates for regenerating tissue, the effectiveness of such collagen based scaffolds for tissue engineering is restricted by generally poor mechanical properties (Vunjak-Novakovic et al., Annu. Rev. Biomed. Eng. 6:131-156 (2004)). Studies by Zmora, et al. have shown that in round pores, such as those created in alginate sponges, cells will aggregate into spherical structures, while the same cells will align with the pore axis in scaffolds with more elongated pore shape (Zmora et al., Biomaterials. 23:4087-4094 (2002)). Furthermore, from a clinical viewpoint, these scaffolds often demonstrate problems with implantability as well as restraining specificity of the scaffold.

### BRIEF DESCRIPTION OF THE DRAWINGS

FIG. 1 is a process diagram of micro-scale fabrication.

FIGS. 2A-2D are SEM micrographs of collagen microthreads. FIG. 2A shows a control sample; FIG. 2B at -20° C.; FIG. 2C at -80° C.; and FIG. 2D at −196° C. White boxes highlighting regions of interest in low magnification (100×) insets indicate field of view in high magnification (700×) images. All processing conditions (in FIGS. 2B, 2C, and 2D) produced microthreads with distinctly different surface topographies from that of the control microthreads in FIG. 2A. Scale bars in high magnification images =20 μm.

FIG. 3 is a diagram showing parameters of interest and their relationship in materials characterization.

### DETAILED DESCRIPTION

One of the fundamental components of tissue engineering—the manipulation of the fate and function of both implanted and native cells—has historically been achieved through the regulation of tissue-specific extracellular matrix ligands and soluble factors. However, since tissue structure and function are interdependent, it is essential that a biomaterial scaffold for tissue engineering possess the ability to present physical cues (via micromechanics, scaffold porosity, and topography) as well as biochemical signaling to cells. Further, there is evidence that the response of a cell to soluble factors is in fact governed by the binding interactions between that cell and the surrounding matrix (Bhatia and Chen, Biomed. Dev. 2:131-144 (1999)), suggesting that these two types of signaling stimuli are interrelated.

The inventors have found that by using phase separation techniques, they can create textured surface features on biopolymer microthread scaffolds. Such textured features increase the surface area of the microthread scaffolds and promote cell migration, orientation, adhesion and cytoskeletal organization. Although phase separation typically produces unordered topographies, the inventors have shown that application of phase separation techniques to the highly anisotropic molecules comprising biopolymer microthreads, phase separation techniques can produce aligned features, resulting in a porous three-dimensional scaffold with aligned surface topographies. More specifically, the inventors have found that different surface topographies can be obtained by freezing the microthreads at different freezing temperatures. Because modifying the freezing rate results in differences in ice crystal nucleation, the aligned surface topographies can be systematically varied by modifying the freezing rate.

Such anisotropic micro- and nano-topographies (i.e. ridges and grooves) have been shown to promote cellular orientation, migration, and cytoskeletal organization. While the width, pitch, and depth of channels can have significant specific effects, it has been reported that such topographies generally stimulate morphological changes and may also induce modification in gene expression (Lim and Donahue, Tissue Eng. 13:1879-1891 (2007)). By creating three dimensional scaffolds with complex hierarchical structure and by studying the ways in which cells interact at all size scales, we can begin to develop biomaterial scaffolds with improved biocompatibility and biofunctionality that effectively mimic the native ECM, control molecular transport, provide signaling cues for tissue regeneration, promote cell growth, and direct cell function as well as new matrix deposition.

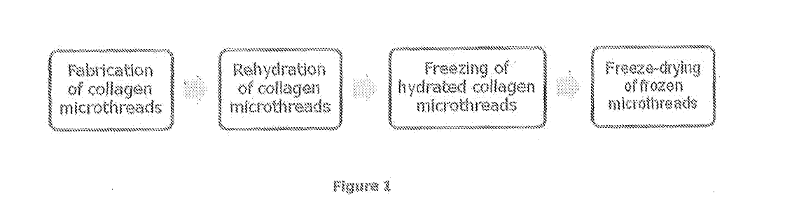

Described herein is a process to fabricate biopolymer threads with surface topographies (see FIG. 1). Biopolymer microthreads are prepared, for example as described (Pins and Silver, Mater. Sci. Eng. 3C:101-107 (1995)). These threads are rehydrated to create a biphasic hydrogel system. By freezing these structures at different temperatures, ice crystals having varying morphologies are formed throughout the microthread. Finally, the frozen microthreads are freeze-dried, sublimating the ice from the frozen thread and leaving a scaffold with complex surface topographies.

The present invention features biopolymer microthreads with surface topographies and compositions that include them. Naturally derived polymer hydrogels have been characterized for use in several tissue engineering applications because they have macromolecular properties similar to—or in some cases, identical to—the native ECM (Drury and Mooney, Biomaterials. 24:4337-4351 (2003)). Materials such as collagen, fibrin, alginate, chitosan, and silk demonstrate highly desirable intrinsic biocompatibility and biofunctionality, and these materials are among those useful in the present compositions and methods.

As noted, the textured microthreads of the invention can include a polymer. In some embodiments, the polymer is a biological polymer, i.e., a polymer isolated from a organism or a recombinant or synthesized polymer having a structure, e.g., amino acid sequence, similar to that found in a naturally-occurring polymer. Any of a wide range of biological polymers can be used to form the microthreads. For example, the polymer can be a naturally occurring polymer such as a proteoglycan, a polypeptide, or glycoprotein, or a carbohydrate or polysaccharide. More specifically, the proteoglycan can be heparin sulfate, chondroitin sulfate, or keratin sulfate; the polypeptide or glycoprotein can be collagen, silk, fibrinogen, elastin, tropoelastin, fibrin, fibronectin, gelatin; and the carbohydrate or polysaccharide can be hyaluronan, a starch, alginate, pectin, cellulose, chitin, or chitosan. The microthreads can be fabricated with only a single type of polymer, e.g., collagen or fibrinogen, two or more kinds of the same type of polymer, e.g., two different polypetides, e.g., collagen and fibrinogen, or two different proteoglycans or two different carbohydrates, or can be fabricated with combination of polymers, i.e., any combination of a proteoglycan, a polypeptide or glycoprotein, and a carbohydrate or polysaccharide. For example, microthreads can be fabricated with collagen and proteoglycans and/or glycoproteins, e.g., collagen and chondroitin sulfate proteoglycan.

The terms “polypeptide” and “peptide” are used herein to refer to a compound of two or more subunit amino acids, amino acid analogs, or other peptidomimetics, regardless of post-translational modification (e.g., amidation, phosphorylation or glycosylation). The subunits can be linked by peptide bonds or other bonds such as, for example, ester or ether bonds. The term “amino acid” refers to natural and/or unnatural or synthetic amino acids, which may, as noted above, be D- or L-form optical isomers. Full-length proteins, analogs, mutants, and fragments thereof are encompassed by this definition.

An exemplary polypeptide is collagen, the major insoluble fibrous protein in the extracellular matrix and in connective tissue. The collagen can be any form of collagen including a fibrillar collagen (e.g., type I, type II, type III or type V; a fibril-associated collagen (e.g., type IV or type IX) or a sheet-forming collagen (e.g., type IV). Fibrillar collagen molecules pack together to form long thin fibrils of similar structure (“tropocollagen”). Type IV, in contrast, forms a two-dimensional reticulum; several other types associate with fibril-type collagens, linking them to each other or to other matrix components. The native collagen fibril is a bundle of many subfibrils, each of which is in turn a bundle of microfibrils. A microfibril is composed of helically coiled collagen molecules each composed of three helical polypeptide chains, The tropocollagen or “collagen molecule” is a subunit of larger collagen aggregates such as fibrils. It is approximately 300 nm long and 1.5 nm in diameter, made up of three polypeptide strands (called alpha chains), each possessing the conformation of a left-handed helix. These three left-handed helices are twisted together into a right-handed coiled coil, a triple helix or “super helix”, a cooperative quaternary structure stabilized by numerous hydrogen bonds. Each triple-helix associates into a right-handed super-super-coil that is referred to as the collagen microfibril.

A distinctive feature of collagen is the regular arrangement of amino acids in each of the three chains of these collagen subunits. The sequence often follows the pattern Gly-Pro-X or Gly-X-Hyp, where X may be any of various other amino acid residues. Proline or hydroxyproline constitute about ⅙ of the total sequence. Exemplary collagen amino acid sequences include, without limitation, collagen alpha-1(I) chain preproprotein: Genbank accession number NP_{—}000079.2, public GI:110349772; collagen alpha-1(II) chain isoform 1 precursor: Genbank accession number NP_{—}001835.3, public GI:111118976; collagen alpha-1(III) chain preproprotein: Genbank accession number NP_{—}000081.1 public GI:4502951.

In some embodiments, the biopolymer can be fibrin, a proteolytic cleavage product of fibrinogen. Fibrinogen, a soluble protein typically present in human blood plasma at concentrations between about 2.5 and 3.0 g/L, is intimately involved in a number of physiological processes including hemostasis, angiogenesis, inflammation and wound healing. Fibrinogen is 340,000 Da hexameric glycoprotein composed of pairs of three different subunit polypeptides, Aα, Bβ, and γ, linked together by a total of 29 disulfide bonds.

Any form of a biopolymer that retains the ability to function (e.g., retains sufficient activity to be used for one or more of the purposes described herein) may be used in the manufacture of the textured microthreads. The biopolymer can be, for example, human collagen or fibrinogen or collagen or fibrinogen of a non-human primate, a domesticated animal, or a rodent. The biopolymer is obtained from a naturally occurring source or is recombinantly produced. The amino acid sequence of the polypeptides can be identical to a standard reference sequence in the public domain. As noted, the present invention includes biologically active variants of polypeptides, and these variants can have or can include, for example, an amino acid sequence that differs from a reference fragment of, for example, a collagen or a fibrinogen polypeptide by virtue of containing one or more mutations (e.g., an addition, deletion, or substitution mutation or a combination of such mutations). One or more of the substitution mutations can be a substitution (e.g., a conservative amino acid substitution), with the proviso that at least or about 50% of the amino acid residues of the variant are identical to residues in the corresponding wild-type fragment of the polypeptides. For example, a biologically active variant of, for example, a collagen or fibrinogen polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a collagen or fibrinogen polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. Alternatively, any of the components can contain mutations such as deletions, additions, or substitutions. All that is required is that the variant polypeptide have at least 5% (e.g., 10%, 20%, 30%, 40%, 50%, 60%, 70%, 80%, 90%, 95%, 99%, 100%, or even more) of the ability of the polypeptide containing only the reference sequences to form microthreads and that those microthreads retain, or substantially retain, the capacity to support cell attachment and proliferation.

The polypeptides may be obtained from any of a wide range of species. It is not necessary that the polypeptides be from a species that is identical to the host, but should simply be amenable to being remodeled by invading or infiltrating cells such as differentiated cells of the relevant host tissue, stem cells such as mesenchymal stem cells, or progenitor cells. The polypeptides useful for the invention can optionally be made from a recipient's own tissue. Furthermore; while the polypeptides will generally have been made from one or more individuals of the same species as the recipient of the microthreads, this is not necessarily the case. Thus, for example, the collagen or fibrinogen can be derived from bovine tissue and be used to make microthreads that can be implanted in a human patient. Species that can serve as recipients of microthreads and polypeptide donors for the production of microthreads can include, without limitation, mammals, such as humans, non-human primates (e.g., monkeys, baboons, or chimpanzees), pigs, cows, horses, goats, sheep, dogs, cats, rabbits, guinea pigs, gerbils, hamsters, rats, or mice.

The polypetides may be partially or substantially pure. The term “substantially pure” with respect to collagen or fibrinogen refers to collagen or fibrinogen that has been separated from cellular components by which it is naturally accompanied, such that it is at least 60% (e.g., 70%, 80%, 90%, 95%, or 99%), by weight, free from polypeptides and naturally-occurring organic molecules with which it is naturally associated. In general, a substantially pure polypeptide will yield a single major band on a non-reducing polyacrylamide gel. A substantially pure polypeptide provided herein can be obtained by, for example, extraction from a natural source (e.g., blood or blood plasma from human or animal sources; e.g., non-human primates (e.g., monkeys, baboons, or chimpanzees), pigs, cows, horses, goats, sheep, dogs, cats, rabbits, guinea pigs, gerbils, hamsters, rats, or mice), chemical synthesis, or by recombinant production in a host cell.

The polypeptides can include post-translational modifications, i.e., chemical modification of the polypeptide after its synthesis. Chemical modifications can be naturally occurring modifications made in vivo following translation of the mRNA encoding the fibrinogen polypeptide subunits or synthetic modifications made in vitro. A polypeptide can include one or more post-translational modifications, in any combination of naturally occurring, i.e., in vivo, and synthetic modifications made in vitro. Examples of post-translational modifications glycosylation, e.g., addition of a glycosyl group to either asparagine, hydroxylysine, serine or threonine residues to generate a glycoprotein or glycopeptides. Glycosylation is typically classified based on the amino acid through which the saccharide linkage occurs and can include: N-linked glycosylation to the amide nitrogen of asparagines side chains, O-linked glycosylation to the hydroxyl oxygen of serine and threonine side chains, and C-mannosylation. Other examples of post-translation modification include, but are not limited to, acetylation, e.g., the addition of an acetyl group, typically at the N-terminus of a polypeptide; alkylation, e.g., the addition of an alkyl group; isoprenylation, e.g., the addition of an isoprenoid group; lipoylation, e.g. attachment of a lipoate moeity; phosphorylation, e.g., addition of a phosphate group to serine, tyrosine, threonine or histidine; and biotinylation, e.g., acylation of lysine or other reactive amino acid residues with a biotin molecule.

The polypeptides can be purified using any standard method know to those of skill in the art including, without limitation, methods based on fibrinogen's low solubility in various solvents, its isoelectric point, fractionation, centrifugation, and chromatography, e.g., gel filtration, ion exchange chromatography, reverse-phase HPLC and immunoaffinity purification. Partially or substantially purified polypeptides can also be obtained from commercial sources, including for example Sigma, St. Louis, Mo.; Hematologic Technologies, Inc. Essex Junction, Vt.; Aniara Corp. Mason, Ohio.

The polypetides can also be produced by recombinant DNA techniques, which are well known in the art. Nucleic acid segments encoding, for example, collagen or fibrinogen polypetides are readily available in the public domain. The terms “nucleic acid” and “polynucleotide” are used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs. The nucleic acid molecules can be synthesized (for example, by phosphoramidite based synthesis) or obtained from a biological cell, such as the cell of a mammal. The nucleic acids can be those of mammal, e.g., humans, a non-human primates, cattle, horses, pigs, sheep, goats, dogs, cats, rabbits, guinea pigs, hamsters, rats, or mice.

An “isolated” nucleic acid can be, for example, a naturally-occurring DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus, or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid. Isolated nucleic acid molecules can be produced by standard techniques.

In some embodiments, the textured microthreads can include a synthetic polymer, for example an aliphatic polyester, a poly(amino acid), poly(propylene fumarate), a copoly(ether-ester), a polyalkylene oxalate, a polyamide, a tyrosine-derived polycarbonate, a poly(iminocarbonate), a polyorthoester, a polyoxaester, a polyamidoestcr, a polyoxaester containing one or more amine groups, a poly(anhydride), a polyphosphazine, or a polyurethane. Wherein an aliphatic polyester is used, it can be a homopolymer or copolymer of: lactides; glycolides; c -caprolactone; hydroxybuterate; hydroxyvalerate; 1,4-dioxepan-2-one; 1,5,8,12-tetraoxy-acyclotetradecane-7,14-dione; 1,5-dioxepan-2-one; 6,6-dimethyl-1,4-dioxan-2-one; 2,5-diketomorpholine; p-dioxanone (1,4-dioxan-2-one); trimethylene carbonate (1,3-dioxan-2-one); alkyl derivatives of trimethylene carbonate; δ-valerolactone; β-butyrolactone; γ-butyrolactone, E-decalactone, pivalolactone, α-diethylpropiolactone, ethylene carbonate, ethylene oxalate; 3-methyl-1,4-dioxane-2,5-dione; 3,3-diethyl-1,4-dioxan-2,5-dione; or 6,8-dioxabicycloctane-7-one.

In some embodiments, the textured microthreads can include a combination of a biopolymer and a synthetic polymer.

Therapeutic agents that aid tissue regeneration can be included in the microthread compositions. These agents can include growth factors including cytokines and interleukins, extracellular matrix proteins and/or biologically active fragments thereof (e.g., RGD-containing peptides), blood and serum proteins, nucleic acids, hormones, vitamins, chemotherapeutics, antibiotics and cells. These agents can be incorporated into the compositions prior to the compositions being placed in the subject. Alternatively, they can be injected into or applied to the composition already in place in a subject. These agents can be administered singly or in combination. For example, a composition can be used to deliver cells, growth factors and small molecule therapeutics concurrently, or to deliver cells plus growth factors, or cells plus small molecule therapeutics, or growth factors plus small molecule therapeutics.

Growth factors that can be incorporated into the biocompatible tissue repair composition include any of a wide range of cell growth factors, angiogenic factors, differentiation factors, cytokines, hormones, and chemokines known in the art. Growth factors can be polypeptides that include the entire amino acid sequence of a growth factor, a peptide that corresponds to only a segment of the amino acid sequence of the native growth factor, or a peptide that derived from the native sequence that retains the bioactive properties of the native growth factor. The growth factor can be a cytokine or interleukin. Any combination of two or more of the factors can be administered to a subject by any of the means recited below. Examples of relevant factors include vascular endothelial cell growth factors (VEGF) (e.g., VEGF A, B, C, D, and E), platelet-derived growth factor (PDGF), insulin-like growth factor (IGF) I and IGF-II, interferons (IFN) (e.g., IFN-α.β, or γ), fibroblast growth factors (FGF) (e.g., FGF1, FGF-2, FGF-3, FGF-4-FGF-10), epidermal growth factor, keratinocyte growth factor, transforming growth factors (TGF) (e.g., TGFα or β), tumor necrosis factor-.alpha., an interleukin (IL) (e.g., IL-1, IL-2, Il-17-IL-18), Osterix, Hedgehogs (e.g., sonic or desert), SOX9, bone morphogenetic proteins (BMP's), in particular, BMP 2, 4, 6, and (BMP-7 is also called OP-1), parathyroid hormone, calcitonin prostaglandins, or ascorbic acid.

Therapeutic agents that are proteins can also be delivered to a recipient subject by administering to the subject, in association with the textured microthreads, cells that have been engineered to express the therapeutic protein. For example, using standard molecular biology techniques, the cell can include an expression vector (e.g., a plasmid or viral vector) containing nucleic acid sequences encoding any one or more of the proteinaceous therapeutic agents exemplified herein. Such transfected or transduced cells will preferably be derived from, or histocompatible with, the recipient. However, it is possible that only short exposure to the factor is required and thus histo-incompatible cells can also be used.

Other useful proteins can include, without limitation, hormones, antibodies, extracellular matrix proteins, and/or biologically active fragments thereof (e.g., RGD-containing peptides) or other blood and serum proteins (e.g., fibronectin, albumin, thrombospondin, van Willebrand factor and fibulin).

Where cells are associated with the textured microthreads for administration to a patient, they cells can be derived from the intended recipient (i.e., patient), an allogeneic donor, or another source. Cell types with which the present compositions can be populated include, but are not limited to, embryonic stem cells (ESC), adult or embryonic mesenchymal stem cells (MSC), monocytes, hematopoetic stem cells, gingival epithelial cells (thus, the present compositions and methods are useful in dental implants and the treatment of periodontal disease), endothelial cells, fibroblasts, or periodontal ligament stem cells, prochondroblasts, chondroblasts, chondrocytes, pro-osteoblasts, osteocytes, or osteoclast. Any combination of two or more of these cell types (e.g., two, three, four, five, six, seven, eight, nine, or ten) may be used in association with the textured microthreads, and methods for isolating and maintaining specific cell types are well-known in the art. The associated cells may be referred to as “donor” cells, and these cells may be used directly after harvest or after being cultured in vitro using standard tissue culture techniques. Donor cells can be infused into, injected into, or otherwise brought into association with the textured microthreads in situ prior to placing of the microthreads or constructs containing them in a mammalian subject. Donor cells can also be cocultured with the textured microthreads using standard tissue culture methods known to those in the art.

As noted, small molecule drugs (e.g., small organic compounds) can also be incorporated and used in the methods of the present invention. This facilitates localized drug delivery (to, for example, a wound (e.g., ischemic tissue) or tumor). Incorporation of antimicrobial agents into the present compositions can provide local high concentrations of antibiotics, thus minimizing the risk of adverse effects associated with long term high systemic doses. An antimicrobial agent can be an antibiotic, and examples of antibiotics useful in the present invention include, without limitation, any representative classes of antibiotics, e.g., 1) aminoglycosides, such as gentamycin, kanamycin, neomycin, streptomycin or tobramycin; 2) cephalosporins, such as cefaclor, cefadroxil or cefotaxime; 3) macrolides, such as azithromycin, clarithromycin, or erythromycin; 4) penicillins, such as amoxicillin, carbenicillin or penicillin; 5) peptides, such as bacitracin, polymixin B or vancomycin; 6) quinolones, such as ciprofloxacin, levofloxacin, or enoxacin; 7) sulfonamides, such as sulfamethazole, sulfacetimide; or sulfamethoxazole; 8) tetracyclines, such as doxycycline, minocycline or tetracycline; 8) other antibiotics with diverse mechanisms of action such as rifampin, chloramphenicol, or nitrofuratoin. Other antimicrobial agents, e.g., antifungal agents and antiviral agents can also be included in the compositions.

Chemotherapeutic agents can also be included in the compositions. Malignant tumors that occur in soft tissue, including for example, tumors of the esophagus, stomach, colon, bladder are typically treated by tumor resection and systemic administration of anticancer drugs. Incorporation of anticancer agents into the biocompatible tissue repair compositions can provide local high concentrations of chemotherapy, thus mitigating the toxicity associated with long term high systemic doses. Examples of classes of chemotherapeutic agents include, without limitation, 1) alkylating agents, e.g., cyclophosphamide; 2) anthracyclines, e.g., daunorubicin, doxorubicin; 3) cycloskeletal disruptors, e.g., paclitaxel; 4) topoisomerase inhibitors, e.g., etoposide; 5) nucleotide analogues, e.g., azacitidine, fluorouracil, gemcitabine; 6) peptides, e.g., bleomycin; 7) platinum-based agents, e.g., carboplatin, cisplatin; 8) retinoids, e.g., all-trans retinoic acid; and 9) vinca alkaloids, e.g., vinblastine or vincristine.

The textured microthreads provided herein are made by methods that include drying and rehydrating a microthread, freezing the rehydrated microthread and then lyophilizing the frozen microthread.

Any of the methods known in the art can be used to fabricate the microthreads. For example, one can use the process taught in U.S. Application Publication Nos.: 2011/0034388 A1 and 2011/0034867 A1, both of which are incorporated herein by reference in their entirety.

In brief, the steps involved in the production of microthreads generally include preparing a biopolymer solution, extruding the solution through an orifice into an aqueous buffered medium, incubating the extruded solution until filament formation is observed, and then drying the filaments. The details of the method can vary depending on the particular kind of biopolymer that is used. For some biopolymers, e.g., collagen, the isolated collagen molecules can be used directly for microthread formation. The collagen is extracted, solubilized and extruded through an orifice to form the microthread. Other biopolymers may be provided as a precursor and cleaved to their active form during preparation of the microthreads. For example, fibrin microthreads are formed by coextruding a solution of fibrinogen, the fibrin precursor, with one or more molecules capable of forming fibrin, under conditions suitable for fibrin formation, into an aqueous buffered medium, incubating the extruded solution until filament formation is observed, and then drying the filaments. During the extrusion process, the fibrinogen is cleaved to generate fibrin monomers that self-assemble in situ to form filaments.

Fibrinogen cleavage can be carried out by any method know to those of skill in the art. The fibrinogen can be suspended in any aqueous medium that is compatible with the activity of the fibrin-forming enzyme e.g., thrombin. Examples of suitable buffer systems include HEPES-buffered saline, tris-buffered saline, phosphate buffered saline, MES, PIPES. Any concentration of fibrinogen that results in fibrin microthread formation can be used. The exact concentration may vary according to extrusion conditions. Suitable concentrations are about 70 mg/mL. “About” indicates that the fibrinogen concentration can vary by up to 10% above or below the recited value. The thrombin can be suspended in any aqueous medium that is compatible with enzymatic activity. Examples of useful buffer systems include HEPES-buffered saline, tris-buffered saline, phosphate buffered saline, MES, PIPES. The buffer may also include a divalent cation, e.g. CaCl₂. Any concentration of thrombin that results in fibrin microthread formation can be used. The exact concentration may vary according to extrusion conditions. Suitable concentrations are about 6 U/mL. “About” indicates that the thrombin concentration can vary by up to 10% above or below the recited value.

It will be appreciated that the concentrations of biopolymers, the pH of the buffers, and the swelling temperature may be adjusted to achieve optimal microthread formation. For example, biopylmers from different sources, e.g., different mammalian species or different isoforms of fibrinogen from the same species, may require different cleavage conditions in order to synthesize microthreads of requisite tensile strength or tissue regeneration properties.

Any apparatus known to those of skill in the art can be used for extrusion of the biopolymer solutions. A suitable apparatus can include a stabilized crosshead on a threaded rod with a crosshead speed of 4.25 mm/min through a blending applicator tip (Micromedics, Inc., St. Paul, Minn.). The blending applicators can be Luer locked to the two syringes through individual bores and mixed in a needle that is Luer locked to the tip. Where coextrusion is desired, the fibrinogen and thrombin solutions can combined and extruded through polyethylene tubing (BD, Sparks, Md.) into an aqueous buffered bath.

The rate of extrusion can vary according to the type of extrusion apparatus that is employed. The rate of extrusion can be expressed as a “rate ratio”, i.e., the ratio of flow velocity/plotter velocity, where flow velocity is the speed with which the fibrin solution emerges from the tubing and plotter velocity is the speed of the extrusion tubing through the aqueous bath. For example, a rate ratio of 2.0 describes extrusion parameters in which the solution flows out of the tubing twice as fast as the tubing tip moves through the aqueous bath. Useful rate ratios for the apparatus described above can range from about 1.5 to about 6.0, e.g., about 1.6, 1.8, 2.0, 2.5, 3.0, 3.5, 4.0, 4.5, 5.0.

The diameter of the tubing, i.e., the orifice from which the solutions are extruded may also vary. For example, the diameter of the orifice has a diameter can range from about 0.2 μm to about 1,000 μm (e.g., less than about 1000 μm, 500 μm, 250 μm, 200 μm, 150 μm, 100 μm or 50 μm) and more than about 10 μm (e.g., more than about 15 μm, 20 μm, 25 μm, 30 μm or 40 μm). The orifice can have a diameter of about 20 μm to about 100 μm. The orifice can have a diameter of about 380 μm. The extruding step is carried out at a temperature between about 25° C. and about 42° C., inclusive.

The nature of the buffer solution, the pH and the temperature of the aqueous bath may also vary. In general, the aqueous bath can be any include any buffer system that is compatible with fibrin polymerization, e.g., HEPES-buffered saline, tris-buffered saline, phosphate buffered saline, MES, PIPES. The pH of the bath can vary from less than about 8.5 (e.g., less than about 8.3, 8.1, 8.0, 7.9, 7.8, 7.7, 7.6, 7.5, 7.4, 7.3, 7.2, 7.1, 7.0, 6.9, 6.8, 6.7) to more than about 5.5 (e.g. more than about 5.7, 5.8, 6.0, 6.1, 6.2, 6.3, 6.4, 6.5, 6.6, 6.7, 6.8). “About” indicates that the pH can vary by up to 0.2 pH units above or below the recited value. Thus, a pH of “about” 7.4, can include, for example, pH 7.2, 7.3, 7.5 or 7.6. The temperature of the bath can be any temperature compatible with fibrin polymerization and can vary from less than about 40° C. (e.g., less than about 38° C., 37° C., 36° C., 35° C., 34° C., 33° C., 32° C., 31° C., 30° C., 29° C., 28° C.) to more than about 18° C. (e.g., more than about 19° C., 20° C., 21° C., 22° C., 23° C., 24° C., 25° C., 26° C., 27° C.).

The incubation step includes features that prevent the extruded solution from adhering or substantially adhering to the surface of the vessel in which the aqueous bath is contained. Any method that is compatible with fibrin polymerization may be used. For example, the vessel can include one or more materials having an extremely low coefficient of friction to provide a non-stick surface, e.g, polytetrafluoroethylene (Teflon.®.), fluorinated ethylene-propylene (FEP) and perfluoroalkoxy polymer resin (PFA). Alternatively or in addition, the aqueous buffered medium can include one or more surfactants, detergents or emulsifying agents, for example, Pluronic.®. surfactants (BASF) polyethylene glycol, or tri-ethylene glycol. The appropriate concentration of such reagent will vary according to the nature of the reagent and may be readily determined empirically by one of skill in the art. Alternatively or in addition, the medium is physically agitated.

Formation of the microthreads in the aqueous bath can typically be observed within a few minutes of the coextrusion process. The incubation step can vary from more than about 1 minute (e.g., 1.5 minutes, 2.0 minutes, 2.5 minutes, 3.0 minutes, 3.5 minutes) to less than about 3.0 hours (e.g. 2.5 hours, 2.0 hours, 1.5 hours, 1.0 hours, 0.5 hours.).

Regardless of the biopolymer that is used and how the microthreads are fabricated, the microthreads are recovered from the medium and permitted to dry. The microthreads can be dried by any method known in the art that will result in the retention of biological and physical functions of the microthreads. Drying methods include, without limitation, e.g., air drying, drying in atmosphere of, or under a stream of, inert gas (e.g., nitrogen or argon). The drying temperature may be ambient temperature, e.g., about 25° C. or it can be a temperature that is mildly elevated relative to ambient temperature, e.g., 28° C., 29° C., 30° C., 31° C., 32° C., 33° C., 34° C., 35° C., 36° C., 37° C., 38° C., 39° C., 40° C., 41° C., 42° C., 43° C. or 44° C.

The rehydration step can be carried out in water or an aqueous medium such as phosphate buffered saline or cell culture medium. Optionally, the rehydration solution may contain a surfactant, e.g., Tween-20 or DMSO or an indicator dye. For ease of handling, the microthreads may be mounted under tension on a frame, e.g., a PDMS frame. The microthreads can be rehydrated in any container; useful containers include those that are configured, e.g., aluminum containers, so that they can be transferred to freezing temperatures once the threads have been rehydrated. The length of the rehydration can vary depending upon many factors, including, for example, the particular biopolymer, the size of the microthreads, the specific rehydration medium and the temperature at which the rehydration is performed, but generally the microthreads are incubated in the rehydration medium have swollen to substantially the same diameter that they had prior to the drying step. In some embodiments the rehydration is carried out until no further increase in diameter can be detected. For example, the rehydration step may range in length from about 10 minutes to about 12 hours or more, e.g., 10 minutes, 20 minutes, 30 minutes, 40 minutes, 50 minutes, 60 minutes or 2 hours, 3 hours, 4 hours, 5 hours, 6 hours, 7 hours, 8 hours, 9 hours, 10 hours, 11, hours, 12 hours or more.

Following the rehydration step, the rehydrated microthreads are freeze-dried. The freezing conditions can vary widely, but the rehydrated micro threads should be frozen at a temperature and for a time that is sufficient to result in the production of surface texturing. The freezing temperature can vary over a wide range e.g., from about −20° C. to about −200° C., for example −40° C., −60° C., −80° C., −100° C., −120° C., −140° C., −160° C., −180° C., −190° C., −196° C., −200° C., −220° C., −240° C. or more. The freezing step can be carried out using any freezing equipment know in the art, e.g., a standard controlled temperature commercial or laboratory freezer, e.g. a −20° C. or −80° C. freezer, or a walk-in freezer. Alternatively the freezing step can be carried out with dry ice and methanol or liquid nitrogen e.g., −196° C. In some embodiments, the microthreads may be flash-frozen in liquid nitrogen and then transferred to −80° C. The freezing period can be for at least 0.25 hours to at least 24 hours or more, e.g., at least 1, at least 2, at least 3, at least 4, at least 5, at least 6, at least 7, at least 8, at least 9, at least 10, at least 11, at least 12, at least 15, at least 18, at least 21 or at least 24 hours.

The microthreads are lyophilized, i.e., freeze-dried. Freeze-drying is a routine technique used in the art (see, for example, U.S. Pat. Nos: 4,619,257; 4,676,070; 4,799,361; 4,865,871; 4,964,280; 5,024,838; 5,044,165; 5,154,007; 6,194,136; 5,336,616; 5,364,756; and 5,780,295, the disclosures of all of which are incorporated herein by reference in their entirety) and suitable equipment is available from commercial sources such as Labconco (Kansas City, Mich., USA). Freeze-drying involves the removal of water or other solvent from a frozen product by a process called sublimation. Sublimation occurs when a frozen liquid (solid) goes directly to the gaseous state without passing through the liquid phase. Those skilled in the art are well aware of the different freeze-drying methodologies available in the art [see, e.g., “A Guide to Freeze-drying for the Laboratory”-an industry service publication by Labconco, (2004); and Franks (1994) Proc. Inst. Refrigeration. 91: 32-39]. Freeze-drying may be accomplished by any of a variety of methods, including, for example, the manifold, batch, or bulk methods.

The lyophilization step can be carried out using any standard method that results in the production of textured microthreads, using for example, a rotary evaporator freeze-drier, a manifold freeze-drier, or a tray freeze-drier. The final water content of the textured microthreads may vary but can be between about 1% and about 10% or more. In one embodiment, the rehydrated threads are placed in an aluminum block, which is frozen and then the entire block is transferred to a shelf in the lyophilizer and held at a temperature of −45° C. After two hours, the vacuum in the lyophilizer is lowered to 50 mtorr and the shelf temperature is ramped to 0° C. The threads are lyophilized under these conditions for 12-24 hours. The lyophylization period can be for at least 1 hour to at least 24 hours or more, e.g., at least 1, at least 2, at least 3, at least 4, at least 5, at least 6, at least 7, at least 8, at least 9, at least 10, at least 11, at least 12, at least 15, at least 18, at least 21 or at least 24 hours.

After lyophilization, the textured threads can be removed from their containers and stored in a desiccator until use. Optionally, the textured microthreads may be mechanically stretched to further align and compress the fibrils. The stretching step can be performed before the freezing and lyophilyzing steps. The amount of stretching can vary, but generally any stretching protocol that results in the formation of textured microthreads can be used. The microthreads can bestretched for example, between 10% and 200% of their resting length. In some embodiments, the textured microthreads can be subjected to repeated cycles of stretching and freezing. The biopolymer concentration can also be varied. For example, modulating the concentration of collagen can affect the packing density of collagen molecules in the collagen microthread.

In some embodiments, the textured microthreads can be chemically cross-linked (e.g. covalently linked) to itself and/or other textured microthreads. Cross-linking can be performed at any point in the preparation of the textured microthreads, for example, before drying, after rehydration or after lyophilization. One suitable method of cross-linking is exposure to ultra-violet (uv) light. Methods for uv cross-linking are well known to those of skill in the art. Levels of uv exposure may vary according to the size and configuration of the textured microthreads and can range for example, from a calculated total energy of about 4 to about 100 J/cm², e.g, about 4.5, 5.0, 8.0, 10.0, 15.0 17.1, 20.0 25.0, 30.0 40.0 50.0 60.0. 70.0 80.0, 90.0, 100.0 J/cm². Cross-linking can also be carried out using chemical cross-linking agents. Chemical cross-linking agents can be homo-bifunctional (the same chemical reaction takes place at each end of the linker) or hetero-bifunctional (different chemical reactions take place at the ends of the linker). The chemistries available for such linking reactions include, but are not limited to, reactivity with sulfhydryl, amino, carboxyl, diol, aldehyde, ketone, or other reactive groups using electrophilic or nucleophilic chemistries, as well as photochemical cross-linkers using alkyl or aromatic azido or carbonyl radicals. Examples of chemical cross-linking agents include, without limitation, glutaraldehyde, carbodiimides, bisdiazobenzidine, and N-maleimidobenzoyl-N-hydroxysuccinimide ester or transglutaminase (factor XIII) crosslinking. Chemical cross-linkers are widely available from commercial sources (e.g., Pierce Biotechnology (Rockford, Ill.); Invitrogen (Carlsbad, Calif.); Sigma-Aldrich (St. Louis, Mo.); and US Biological (Swampscott, Mass.)). Particularly suitable cross-linking reagents include 1-ethyl-3-[3-dimethylaminopropyl]carbodiimide hydrochloride (EDAC), DHT (dehydrothermal treatment. i.e., heat and vacuum together) and N-hydroxysulfosuccinimide (NHS). These treatments can also be combined (e.g., DHT and EDC together). The duration of the cross-linking reaction may vary according to the cross-linking agent that is used, the reaction temperature and the tensile strength desired.

In some embodiments, that textured microthreads may be stretched and cross-linked. The stretching and crosslinking steps can be carried out in any order that results in the production of a textured microthread.

Optionally, the textured microthreads can be submitted to treatments to diminish the bioburden. This process is expected to decrease the level of infectious microorganisms within the fibrin microthreads. As used herein, a process used to inactivate or kill “substantially all” microorganisms (e.g., bacteria, fungi (including yeasts), and/or viruses) in the fibrin microthreads is a process that reduces the level of microorganisms in the textured microthreads by least 10-fold (e.g., at least: 100-fold; 1,000-fold; 10⁴-fold; 10⁵-fold; 10 ⁶-fold; 10⁷-fold; 10⁸-fold; 10⁹-fold; or even 10¹⁰-fold) compared to the level in the textured microthreads prior to the process. Any standard assay method may be used to determine if the process was successful. These assays can include techniques that directly measure microbial growth, e.g., the culture of swab samples on artificial growth media, or molecular detection methods, such as quantitative PCR.

The textured microthreads can be exposed to gamma-, x-, e-beam, and/or ultra-violet (wavelength of 10 nm to 320 nm, e.g., 50 nm to 320 nm, 100 nm to 320 nm, 150 nm to 320 nm, 180 nm to 320 nm, or 200 nm to 300 nm) radiation in order to decrease the level of, or eliminate, viable bacteria and/or fungi and/or infectious viruses. More important than the dose of radiation that the textured microthreads is exposed to is the dose absorbed by the textured microthreads. While for thicker textured microthreads, the dose absorbed and the exposure dose will generally be close, in thinner textured microthreads the dose of exposure may be higher than the dose absorbed. In addition, if a particular dose of radiation is administered at a low dose rate over a long period of time (e.g., two to 12 hours), more radiation is absorbed than if it is administered at a high dose rate over a short period of time (e.g., 2 seconds to 30 minutes). One of skill in the art will know how to test for whether, for a particular textured microthreads, the dose absorbed is significantly less than the dose to which the fibrin microthreads is exposed and how to account for such a discrepancy in selecting an exposure dose.

The physical properties of the textured microthreads will vary according to size, the biopolymer that is used and the methods used for synthesis. The textured microthreads can have a variety of surface topographies including for example, micron-scale ridges with intervening grooves. The bulk threads can range in diameter from approximately 20-300 microns in diameter. The periodicity and dimensions of the grooves can vary for example, with ridges from about 0.5 microns to about 20 microns in height and about 0.5 microns to about 20 microns in width. In some embodiments, ridges can be about 0.5 microns to about 2.0 microns in height and about 0.5 microns to about 2.0 microns in width. The majority of the ridges, i.e., more than 50% of the ridges may be aligned with one another or substantially parallel to another. In some embodiments, the textured microthreads may also include pores.

The diameter of the textured microthreads may also vary depending upon the size of the orifice from which the solutions are extruded, the polymer that is used. For example, the diameter of the threads can range from about 0.2 μm to about 1,000 μm (e.g., less than about 1000 μm, 500 μm, 250 μm, 200 μm, 150 μm, 100 μm or 50 μm) and more than about 10 μm (e.g., more than about 15 μm, 20 μm, 25 μm, 30 μm or 40 μm). The textured microthreads can have a diameter of about 20 μm to about 100 μm. The textured microthreads can have a diameter of about 380 μm.

In general, the tensile strength of the textured microthreads can range from more than about 0.1 MPa (e.g., 0.2, 0.4, 0.5, 1.0, 2.0, 4.0) to less than about 25 MPa (e.g., 22 MPa, 20 MPa, 18 MPa, 15 MPa, 10 MPa). Methods for measuring tensile strength are well-known to those of skill in the art.

The biological activity of the textured microthreads, e.g., the capacity of to mediate tissue regeneration, can be assayed by any method known to those of skill in the art. Examples include measuring cell ingrowth, cell proliferation, cell orientation and alignment relative to the fibrin microthread axis.

The textured microthreads of the invention can be combined with one or more therapeutic agents. A therapeutic agent, for example, a growth factor, a protein, a chemotherapeutic agent, a vitamin, a mineral, an antimicrobial agent, a small organic molecule, or a biological cell can be added by and (a) extruding the therapeutic agent with polymer and the molecule thereby producing a textured microthread microthread comprising the therapeutic agent or (b) associating the therapeutic agent with a formed microthread or c) associating the therapeutic agent with a textured microthread during or after its preparation. The therapeutic agent can be covalently bonded to the textured microthread. The bonding agent can be a ligase, wherein the ligase generates a carbon-oxygen bond, a carbon-sulfur bond, a carbon-nitrogen bond, or a carbon-carbon bond between the therapeutic agent and the textured microthread.

The textured microthreads can be configured in many forms according to the size and shape of the tissue repair that is desired. The coextrusion step may be repeated one or more times to produce a multifilament textured microthread scaffold. Alternatively the textured microthreads can be assembled into hierarchically organized structures such as woven fabrics or ropes of variable size, shape, and character, which may be used alone or in conjunction with other tissue repair materials such as woven or non-woven meshes, pins, screws, plates, patches, filaments, and natural or mechanical valves. The microthreads may be present, for example, as a reinforcing element. The mechanical properties, surface chemistries and porosities of the microthreads can be varied and controlled to direct, alter, and/or facilitate multidimensional cellular alignment and tissue regeneration.

The compositions, whether used alone or in combination with another repair substance or device can be shaped in the form of a mesh, dressing, gauze, web, film, patch, sheath or graft for application to or implantation in tissue in need of repair. For example textured microthread compositions may be woven or braided or otherwise attached to other polymers or tissue repair compositions. The textured microthread can be combined with a microthread comprising a non-fibrin polymer. Synthetic polymers include the synthetic polymer comprises an aliphatic polyester, a poly(amino acid), poly(propylene fumarate), a copoly(ether-ester), a polyalkylene oxalate, a polyamide, a tyrosine-derived polycarbonate, a poly(iminocarbonate), a polyorthoester, a polyoxaester, a polyamidoester, a polyoxaester containing one or more amine groups, a poly(anhydride), a polyphosphazine, a polyurethane, a biosynthetic polymer, or a combination thereof. The aliphatic polyester comprises homopolymers or copolymers of: lactides; glycolides; .epsilon.-caprolactone; hydroxybuterate; hydroxyvalerate; 1,4-dioxepan-2-one; 1,5,8,12-tetraoxyacyclotetradecane-7,14-dione; 1,5-dioxepan-2-one; 6,6-dimethyl-1,4-dioxan-2-one; 2,5-diketomorpholine; p-dioxanone(1,4-dioxan-2-one); trimethylene carbonate(1,3-dioxan-2-one); alkyl derivatives of trimethylene carbonate; Δ-valerolactone; β-butyrolactone; gamma-butyrolactone, epsilon-decalactone, pivalolactone, α,α-diethylpropiolactone, ethylene carbonate, ethylene oxalate; 3-methyl-1,4-dioxane-2,5-dione; 3,3-diethyl-1,4-dioxan-2,5-dione; 6,8-dioxabicycloctane-7-one; or combinations thereof. Other polymers can include polymers derived from natural sources e.g., collagen and collagen based-compositions. A biosynthetic polymer can include a polymer comprising a sequence found in collagen, elastin, thrombin, fibronectin, a starch, gelatin, alginate, pectin, oxidized cellulose, chitin, chitosan, tropoelastin, hyaluronic acid, a ribonucleic acid, a deoxyridonucleic acid, a polypeptide, a polysaccharide, or a combination thereof or a natural polymer for example collagen or a collagen-based material, hyaluronic acid or a hyaluronic acid-based material, cellulose or a cellulose-based material, silk and combinations thereof.

Alternatively or in addition, such composition may be synthetic in origin. Examples of commercially available polypropylene meshes can include: Marlex.™. (C R Bard, Inc., Cranston, R.I.), Visilex.®. (C R Bard, Inc., Cranston, R.I.), PerFix.®. Plug (C R Bard, Inc., Cranston, R.I.), Kugel.™. Hernia Patch (C R Bard, Inc., Cranston, R.I.), 3DMax.®. (C R Bard, Inc., Cranston, R.I.), Prolene.™, (Ethicon, Inc., Somerville, N.J.), Surgipro.™. (Autosuture, U.S. Surgical, Norwalk, Conn.), Prolite.™. (Atrium Medical Co., Hudson, N.H.), Prolite Ultra.™. (Atrium Medical Co., Hudson, N.H.), Trelex.™. (Meadox Medical, Oakland, N.J.), and Parietene.®. (Sofradim, Trevoux, France). Examples of commercially available polyester meshes include Mersilene.™. (Ethicon, Inc., Somerville, N.J.) and Parietex.®. (Sofradim, Trevoux, France). Examples of commercially available PTFE meshes include Goretex.®. (W. L. Gore & Associates, Newark, Del.), Dualmesh.®. (W. L. Gore & Associates, Newark, Del.), Dualmesh.®. Plus (W. L. Gore & Associates, Newark, Del.), Dulex.®. (C R Bard, Inc., Cranston, R.I.), and Reconix.®. (C R Bard, Inc., Cranston, R.I.).

Other useful compositions include resorbable meshes. Polymers used to make resorbable meshes can include polyglycolic acid (Dexon.™., Syneture.™., U.S. Surgical, Norwalk, Conn.), poly-l-lactic acid, polyglactin 910 (Vicryl.™., Ethicon, Somerville, N.J.), or polyhydroxylalkaoate derivatives such as poly-4-hydroxybutyrate (Tepha, Cambridge, Mass.). Composite meshes, i.e., meshes that include both resorbable and non-resorbable materials can be made either from combinations of the materials described above or from additional materials. Examples of commercially available composite meshes include polypropylene/PTFE: Composix.®. (C R Bard, Inc., Cranston, R.I.), Composix.®. E/X (C R Bard, Inc., Cranston, R.I.), and Ventralex.®. (C R Bard, Inc., Cranston, R.I.); polypropylene/cellulose: Proceed.™. (Ethicon, Inc., Somerville, N.J.); polypropylene/Seprafilm.®.: Sepramesh.®. (Genzyme, Cambridge, Mass.), Sepramesh.®. IP (Genzyme, Cambridge, Mass.); polypropylene/Vicryl: Vypro.™. (Ethicon, Somerville, N.J.), Vypro.™. II (Ethicon, Somerville, N.J.); polypropylene/Monocryl(poliglecaprone): Ultrapro.®. (Ethicon, Somerville, N.J.); and polyester/collagen: Parietex.®. Composite (Sofradim, Trevoux, France).

The step of combining a first textured microthread with a second textured microthread comprising a another polymer can include weaving the first textured microthread and the second textured microthread, bundling the first textured microthread and the second textured microthread to form a filament, or tying or interlacing the first textured microthread and the second textured microthread to form a non-woven mesh, associating the first textured microthread with a substrate or a woven or non-woven mesh, a surgical pin, a surgical screw, a surgical plate, a physiologically acceptable patch, dressing, bandage, or a natural or mechanical valve. The textured microthread compositions may be used in the preparation of a medicament for tissue repair, wherein the tissue repair comprises tissue augmentation or the replacement of all or part of a tissue. The tissue repaired comprises skin, muscle, or a connective tissue.

The biocompatible tissue repair compositions described herein can be used to treat any of a wide range of disorders in which augmentation or repair of tissue is needed. Tissue defects can arise from diverse medical conditions, including, for example, congenital malformations, traumatic injuries, infections, and oncologic resections. Thus, the biocompatible tissue repair compositions can be used to repair defects in any soft tissue (e.g., tissues that connect, support, or surround other structures and organs of the body). Soft tissue can be any non-osseous tissue. For example, soft tissue includes epithelial tissue, which covers the outside of the body and lines the organs and cavities within the body. Examples of epithelial tissue include, but are not limited to, simple squamous epithelia, stratified squamous epithelia, cuboidal epithelia, or columnar epithelia.

Soft tissue can also be connective tissue, which functions to bind and support other tissues. One type of connective tissue that can be treated with the present compositions is loose connective tissue (also known as areolar connective tissue), which binds epithelia to underlying tissues and holds organs in place. It is found in the skin, beneath the dermis; in places that connect epithelium to other tissues; underneath the epithelial tissue of all the body systems that have external openings; within the mucus membranes of the digestive, respiratory, reproductive, and urinary systems; and surrounding the blood vessels and nerves.

Loose connective tissue is named for the loose “weave” of its constituent fibers which include collagenous fibers, elastic fibers (long, thread-like stretchable fibers composed of the protein elastin) and reticular fibers (branched fibers consisting of one or more types of very thin collagen fibers): Connective tissue can also be fibrous connective tissue, such as tendons, which attach muscles to bone, and ligaments, which joint bones together at the joints. Fibrous connective tissue is composed primarily of tightly packed collagenous fibers, an arrangement that maximizes tensile strength. Soft tissue can also be muscle tissue; muscle tissue includes skeletal muscle, which is responsible for voluntary movements; smooth muscle, which is found in the walls of the digestive tract, bladder arteries and other internal organs; and cardiac muscle, which forms the contractile wall of the heart.

The biocompatible tissue repair compositions can be used to repair soft tissues in many different organ systems that fulfill a range of physiological functions in the body. These organ systems can include, but are not limited to, the muscular system, the genitourinary system, the gastroenterological system, the integumentary system, the o circulatory system and the respiratory system. The compositions are particularly useful for repairs to connective tissue, for example, tendons and ligaments. The biocompatible tissue repair compositions are suitable for hernia repair.

The compositions can be used to treat other medical conditions that result from tissue weakness. One condition for which the biocompatible tissue repair compositions are useful is in the repair of organ prolapse. Prolapse is a condition in which an organ, or part of an organ, falls or slips out of place. Prolapse typically results from tissue weakness that can stem from either congenital factors, trauma or disease. Pelvic organ prolapse can include prolapse of one or more organs within the pelvic girdle; tissue weakening due to pregnancy, labor and childbirth is a common cause of the condition in women. Remedies include both non-surgical and surgical options; in severe cases, reconstruction of the tissues of the pelvic floor, i.e., the muscle fibers and connective tissue that span the area underneath the pelvis and provides support for the pelvic organs, e.g., the bladder, lower intestines, and the uterus (in women) may be required.

The biocompatible tissue repair compositions are also useful in repairs of the gastrointestinal system. Esophageal conditions in need of repair include, but are not limited to, traumatic rupture of the esophagus, e.g., Boerhaave syndrome, Mallory-Weiss syndrome, trauma associated with iatrogenic esophageal perforation that may occur as a complication of an endoscopic procedure or insertion of a feeding tube or immolated surgery; repair of congenital esophageal defects, e.g., esophageal atresia; and oncologic esophageal resection.

The compositions can be used to repair tissues that have never been repaired before or they can be used to repair tissues that have been treated one or more times with compositions or with other methods known in the art or they can be used along with other methods of tissue repair including suturing, tissue grafting, or synthetic tissue repair materials.

The compositions can applied to an individual in need of treatment using techniques known to those of skill in the art. The biocompatible tissue repair compositions can be: (a) wrapped around a tissue that is damaged or that contains a defect; (b) placed on the surface of a tissue that is damaged or has a defect; (c) rolled up and inserted into a cavity, gap, or space in the tissue. One or more (e.g., one, two, three, four, five, six, seven, eight, nine, ten, 12, 14, 16, 18, 20, 25, 30, or more) such compositions, stacked or adjacent to each other, can be used at any particular site. The compositions can be held in place by, for example, sutures, staples, tacks, or tissue glues or sealants known in the art (and any of these items can be included in the kits of the invention). Alternatively, if, for example, packed sufficiently tightly into a defect or cavity, they may need no securing device.

The invention features an article of manufacture comprising a measured amount of textured microthreads wherein the textured microthread or the tissue engineering construct is optionally packaged within a sterile container, and one or more of: a package insert comprising instructions for use, an antiseptic agent, a buffered solution, a saline solution, a gel, an ointment, a cream, scissors, a scalpel, a clamp, a needle, a spatula, sutures, gauze, surgical gloves, and a tissue culture vessel.

### EXAMPLES

### Example 1

### Production of Self-Assembled Collagen Microthreads

### Preparation of Acid-Soluble Collagen

Acid-soluble type I collagen was extracted from rat tails as previously described [27, 32]. Tendon fibers were dissected from Sprague-Dawley rats with hemostats, rinsed twice in phosphate buffered saline (PBS), and dissolved in 1600 mL of 3% (vol/vol) acetic acid solution overnight. The solution was refrigerated, centrifuged at 48° C. for 2 hour at 8000 g, and the supernatant retained. A volume of 320 mL of 30% NaCl (wt/vol) solution was slowly dripped into the solution to precipitate the collagen. The entire solution and precipitate were centrifuged at 48° C. for 30 minutes and the supernatant discarded. The pellets were resuspended in 400 mL of 0.6% (vol/vol) acetic acid solution and stirred at 48° C. overnight or until completely dissolved. The solution was dialyzed five times against 4 L of 1 mM HCl, each for at least 4 hour. The resulting collagen solution was lyophilized and stored at 48° C. until use. Prior to extruding threads, lyophilized collagen was dissolved in a rotating vessel overnight at 48° C. in 5 mM HCl, at a final concentration of 10 mg/mL. The collagen solution was degassed by centrifugation before extrusion to remove trapped air bubbles.

### Collagen Microthread Extrusion

Collagen microthreads were extruded using a protocol described previously. Briefly, a syringe pump (KD Scientific, New Hope, Pa.) set at a flow rate of 0.25 mL/min was used to extrude the previously described collagen solution through 0.86 mm inner diameter polyethylene tubing (Becton Dickinson, Franklin, N.J.). The solution was extruded into a bath of fiber formation buffer (pH 7.42, 135 mM NaCl, 30 mM TrizmaBase (Tris), and 5 mM NaPO₄ dibasic; Sigma, St. Louis, Mo.) that controls the polymerization of the collagen solution; the microthreads were incubated in this bath at 37° C. for 24 hours. The buffer was replaced with a fiber incubation buffer (pH 7.42, 135 mM NaCl, 10 mM Tris, and 30 mM sodium phosphate dibasic, Sigma) and the microthreads were incubated in this solution at 37° C. for 24 hours. The incubation buffer was replaced with distilled water, and the microthreads were maintained at 37° C. for an additional 24 hours. Finally, the microthreads were removed from the water bath, air dried, and stored at room temperature in a desiccator until use.

### Fabrication of Porous Collagen Microthreads

A freeze-drying technique was used to create porous collagen microthreads. Microthreads were secured at the ends using silicone adhesive to an aluminum weigh boat. The threads were fully rehydrated with distilled water for 15 minutes before being frozen at one of three experimental freezing temperatures: −20° C., −80° C., or −196° C. (liquid nitrogen). To prevent dehydration of the microthreads to be frozen at −20° C. and −80° C., these weigh boats were quickly transferred to pre-frozen boxes at −20° C. and −80° C., respectively. Liquid nitrogen-frozen microthreads were rehydrated and then immediately submerged in liquid nitrogen for 15 minutes to flash-freeze them. Following this freezing period, liquid nitrogen-frozen microthreads were transferred to −80° C. for 24 hours. Microthreads frozen at −20° C. and −80° C. were maintained at their respective temperatures for 24 hours. Threads and weigh boats were transferred in boxes to a pre-frozen lyophilizer (to prevent unwanted thawing of the threads) for freeze-drying. Following lyophilization, microthreads were stored in a desiccator until use.

### Electron Microscopy

Collagen microthreads were imaged with a scanning electron microscope (SEM) to qualitatively characterize thread morphology, surface topography, and pore structure. Porous and control collagen microthreads were mounted on aluminum stubs (Ted Pella, Redding, Calif.) coated with double-sided carbon tape and sputter coated with a thin layer of gold—palladium at 45 mA for 30 seconds. Images were acquired at 15 kV using a LEO Gemini 982 Field Emission Gun SEM.

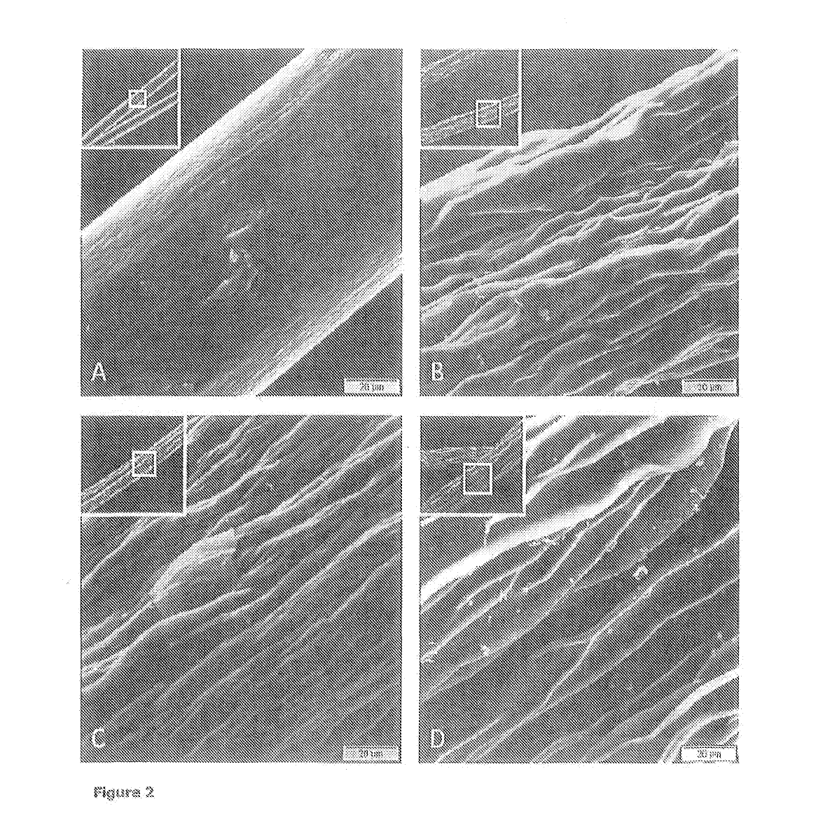

Samples were analyzed using scanning electron microscopy to assess the overall morphology, investigate the existence of porosity, and qualitatively analyze the surface topography of the collagen microthreads. Resulting low (100×, insets) and high (700×) magnification images are shown in FIG. 2. As the figure shows, all microthreads demonstrate some degree of fibrillar alignment. Grooves, ridges, and divots can be seen in all samples and the freeze-dried microthreads appeared larger in diameter than control microthreads. Initial observations reveal the largest scale features in microthreads frozen at −196° C. (Panel D), while the smallest scale features were in the −80° C. microthreads (Panel C). Microthreads created by freezing at −20° C. demonstrated surface topographies with features approximately in the middle.

As the biomaterial scaffold advances, it will become increasingly important that these substrates mimic the biochemical and structural characteristics of the native extracellular matrix to control cell functions in tissue engineering applications. Naturally-derived biopolymer scaffolds in the form of sponges, hydrogels, and films demonstrate advantageous biochemical properties but are limited in their mechanical properties and implantability. While a number of techniques have been used to impart micro- and nanoscale topographical features on model surfaces such as polycaprolactone, polystyrene, and silica, the use of these biomaterials cannot be translated to clinical application because they are planar and cannot be used in vitro or in vivo as templates for tissue engineering. Therefore, there remains a need for a biomaterial scaffold composed of natural biological polymer that whose biochemistry and structure is analogous to that of native extracellular matrix. Furthermore, such a scaffold should demonstrate effective control over cell function in both in vitro and in vivo environments. The current study sought to combine the biochemical and mechanical advantages of collagen microthreads with micro- and nanoscale topographies. Briefly, collagen microthreads were fabricated using previously developed techniques, rehydrated, and freeze-dried to create complex surface topographies observable using scanning electron microscopy. Rehydrated microthreads were frozen at −20° C., −80° C., or −196° C. to generate different ice crystal morphology and thus, different final pore structure.

As shown in FIG. 2, the microthreads that had been rehydrated, frozen and then lyophilized showed extensive aligned and regular submicron features (panels B, C and D) compared to the untrated microthreads (panel A). Processed microthreads (FIG. 2, Panels B-D) possessed surface features including grooves and ridges aligned with the axis of the microthread. Rehydration and subsequent freeze-drying of these microthreads resulted in the expansion of this compacted and aligned collagen network to effectively amplify the surface features observed in standard microthreads.

Microthreads frozen at −80° C. demonstrated finer, smaller-scale features than microthreads frozen at the other temperatures, which may make these scaffolds better suited for applications in tissue engineering. Micro- and nanoscale surfaces are abundant throughout the body and, as previously discussed, the presence of such features on biomaterials scaffolds for tissue engineering enhances the cell-signaling ability of the scaffold. Similarly, the highly aligned nature of the features on the microthreads developed herein may facilitate oriented cell growth and migration, suggesting that these collagen microthread scaffolds may be effective in promoting and directing regeneration of highly aligned tissues such as tendons, ligaments, and muscle.

As previously discussed, studies have shown that substrate features at the submicron scale can have profound effects on cell functions and fate. While the qualitative analysis that was performed in this study identified −80° C. as a good candidate for freezing temperature, the other temperatures should not be excluded. By relating the results of more in-depth materials characterization studies to fabrication parameters and cellular interactivity data, we will be able to create microthread scaffolds that better mimic the extracellular matrix of native aligned tissues.

Changes in the fabrication parameters of the microthreads are expected to affect the morphology of the resulting scaffold.

### Effects of Stretching

Previous work has shown that stretching collagen microthreads results in further alignment and compaction of collagen fibrils, which would thus lead to increased alignment of the amplified surface features after being hydrated, frozen, and freeze-dried.

### Effects of Collagen Concentration

Modulating the concentration of collagen would affect the packing density of collagen molecules in the collagen microthread, which may have effects on the resulting freeze-dried microthread.

### Effects of Crosslinking

By utilizing physical or chemical crosslinking techniques such as DHT and EDC, microthreads with enhanced mechanical strength and stiffness can be created. Previous work has shown that crosslinking microthreads limits their ability to swell when rehydrated, which would then restrict the size of ice crystals being formed within the freezing thread, ultimately changing the surface topography and pore structure.

### Material Characterization

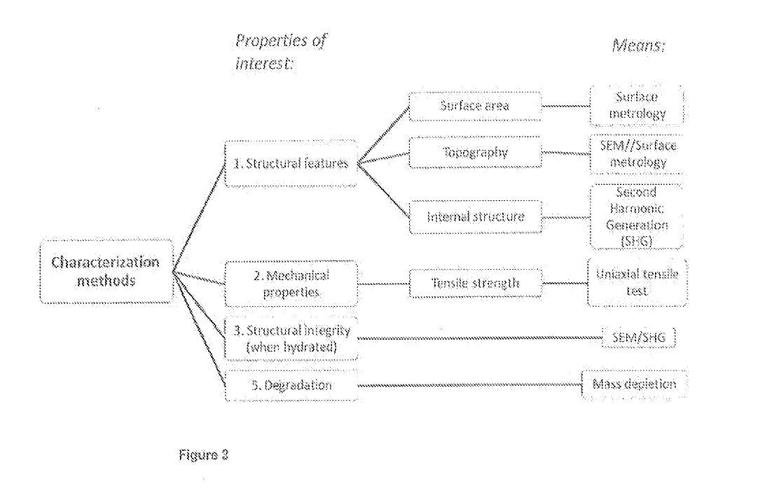

There are several material properties that need to be characterized to validate the methods discussed herein. These include structural features such as surface area, surface topography, and the internal pore structure as well as mechanical properties such as tensile strength. For use as a scaffold for tissue engineering, it will be necessary to rehydrate the microthreads. To remain effective, the distinct features that have been imparted to the microthread scaffolds must be preserved upon rehydration. Finally, the degradation kinematics of the microthreads will need to be assessed. These properties of interest and means to assess these properties are shown in FIG. 3

Preliminary studies have been conducted using surface metrology to quantitatively characterize the surface of the microthreads. Briefly, a scanning white light confocal microscope was used to measure the surface topography of the microthreads and provide data that could allow for the extrapolation of a number of other parameters. The goal of these measurements is to provide quantitative information about the microthreads' surfaces including, but not limited to:
Area-scale relationship (to determine the scale of observation at which the area of a surface approaches a constant),
Topographical feature alignment, and
Correlation of surface parameters to performance parameters such as cell attachment, alignment, and migration.

We have an example of this sort of analysis; a standard collagen microthread was measured with a scanning white light confocal microscope. The cylindrical shape of the microthread is easily identifiable as are aligned and regular surface features. Subsequent analysis of this data would be performed, and the results would be compared to the other processing conditions and would also be correlated to performance parameters such as cell attachment, alignment, and migration.

### Assessment of Cellular Interactivity.

Initial studies investigating the cellular interactivity of these microthreads would involve the use of human dermal fibroblasts. Cellular activity parameters such as attachment, alignment, and migration would be assessed in vitro using methods previously described. The results of these studies could potentially be correlated to processing parameters such as freezing temperature, collagen concentration, crosslinking, and stretching to identify the best fabrication technique.

Applications for the present Microthreads in Biomaterials Research: Considering the prevalence of tendon and ligament injuries and the risks associated with repair and replacement using grafts, there is increasing effort to move towards a biomaterials/tissue engineering approach to regenerating these tissues. However, there still remains a need for a clinically-applicable off-the-shelf regenerative template for aligned tissues that can promote and direct the natural wound healing processes of the body. The microthreads developed could serve as scaffolds for in vitro tissue engineering as well as in vivo tissue regeneration. Similarly, these novel biomaterial scaffolds could also be used for engineering or regenerating other highly aligned tissue such as skeletal or cardiac muscle tissue.

### Example 2

### Mechanical Testing

Ultimate tensile strength (UTS) of the textured microthreads was analyzed using an Instron E-100. Uniaxial load to failure at 50% strain rate in textured collagen microthreads that had been frozen at −80° C. and standard untreated microthreads was compared. There was a statistically significant increase (from 2-4 MPa) in the ultimate tensile strength of textured microthreads that had been frozen −80° C. compared to untreated threads (p=2.02×10⁴). These data showed that the textured microthreads have increased mechanical strength relative to standard microthreads.

Uniaxial load to failure at 50% strain rate in textured fibrin microthreads that had been cross-linked with EDC was compared with uncross-linked textured fibrin microthreads. The cross-linked threads showed a statistically significant increase in UTS.

A number of embodiments of the invention have been described. Nevertheless, it will be understood that various modifications may be made without departing from the spirit and scope of the invention.

What is claimed is:

1. A method of making a textured microthread, the method comprising:
(a) providing a microthread;
(b) drying and subsequently rehydrating the microthread, thereby generating a rehydrated microthread; and
(c) freeze-drying the rehydrated microthread, thereby forming a textured microthread.

2. The method of claim 1, further comprising step (d):
(d) lyophilizing the frozen microthread, thereby exposing the surface of the textured microthread.

3. The method of claim 1, wherein the microthread comprises a naturally occurring polymer.

4. The method of claim 3, wherein the naturally occurring polymer comprises a proteoglycan, a polypeptide, a glycoprotein, a carbohydrate, or a polysaccharide.

5. The method of claim 3, wherein the microthread comprises collagen and a proteoglycan and/or a glycoprotein.

6-9. (canceled)

10. The method of claim 1, further comprising step (e):
(e) associating a therapeutic agent with the surface of the textured microthread.

11. The method of claim 10, wherein the therapeutic agent is a biological cell, a small organic compound, or a biological agent.

12-15. (canceled)

16. A textured microthread made by the method of claim 1.

17. (canceled)

18. A physiologically acceptable composition comprising a textured microthread made by the method of claim 1 and a physiologically acceptable carrier.

19. The physiologically acceptable composition of claim 18, wherein the physiologically acceptable carrier is a solution or gel.

20-21. (canceled)

22. A kit comprising the textured microthread of claim 16, wherein the textured microthread or the tissue engineering construct is optionally packaged within a sterile container, and one or more of: a package insert comprising instructions for use, an antiseptic agent, a buffered solution, a saline solution, a gel, an ointment, a cream, scissors, a scalpel, a clamp, a needle, a spatula, sutures, gauze, surgical gloves, and a tissue culture vessel.

23-27. (canceled)

28. A textured microthread, wherein the microthread comprises a naturally occurring polymer and has a surface comprising micron-scale ridges with intervening grooves.

29. The textured microthread of claim 28, wherein the microthread has a diameter of about 20 microns to about 200 microns.

30. The textured microthread of claim 28, wherein the ridges are about 0.5 microns to about 20 microns in height and about 0.5 microns to about 20 microns in width.

31. (canceled)

32. The textured microthread of claim 28, wherein a majority of the ridges are aligned with respect to one another.

33. The textured microthread of claim 32, wherein the ridges are aligned and oriented with the long axis of the microthread.

34-35. (canceled)

36. The textured microthread of claim 28, wherein the naturally occurring polymer comprises a proteoglycan, a polypeptide, a glycoprotein, a carbohydrate, or a polysaccharide.

37-39. (canceled)

40. The textured microthread of claim 28, wherein the microthread is associated with a therapeutic agent.

41-49. (canceled)

50. The textured microthread of claim 28, further comprising a cladding material, glue, binder, suture, or sleeve, wherein the cladding material, glue, binder, suture or sleeve bundles the microthread together with other microthreads.

51-52. (canceled)

53. A method of treating a patient in need of tissue augmentation or repair, the method comprising administering to the patient a textured microthread of claim 28, wherein the microthread is placed in contact with the tissue in need of augmentation or repair.

54. The method of claim 53, wherein the patient is a human.

55. The method of claim 53, wherein the tissue in need of augmentation or repair comprises skin, muscle, or a connective tissue.
